(19) United States
(12) Patent Application Publication
(10) Pub. No.: US 2023/0286008 A1
(43) Pub. Date: Sep. 14, 2023

(54) DROPLET EJECTOR WITH TREATED SURFACE

(71) Applicant: Pneuma Respiratory, Inc., Boone, NC (US)

(72) Inventors: Charles Eric Hunter, Boone, NC (US); Matthew Culpepper, Boone, NC (US); Michael Scoggin, Boone, NC (US); Caley Modlin, Boone, NC (US); Jeffrey Miller, Boone, NC (US); Jose Salazar, Boone, NC (US); Cristian Salazar, Boone, NC (US); Gregory Rapp, Boone, NC (US); Jacob Greenfield, Boone, NC (US)

(73) Assignee: Pneuma Respiratory, Inc., Boone, NC (US)

(21) Appl. No.: 18/181,258

(22) Filed: Mar. 9, 2023

Related U.S. Application Data
(60) Provisional application No. 63/346,794, filed on May 27, 2022.
(60) Provisional application No. 63/323,770, filed on Mar. 25, 2022.
(60) Provisional application No. 63/318,202, filed on Mar. 9, 2022.

(51) Int. Cl. B05B 17/00; B05B 17/06

(52) U.S. Cl. CPC B05B 17/0646; B05B 17/0661

(57) ABSTRACT

A droplet delivery device includes an ejector mechanism with an ejector mesh or similar substrate with apertures that is treated by one or more of coatings, roughening, metal layer deposition and laser ablation to provide desirable production and size of droplets, such as in an inhaled aerosol from the device.

### CROSS-REFERENCE TO RELATED APPLICATIONS

The present application claims the benefit of priority of Provisional Patent Application No. 63/318,202 entitled “DROPLET EJECTOR WITH OLEOPHOBIC COATING” filed Mar. 9, 2022, Provisional Patent Application No. 63/323,770 entitled “ROUGHENED DROPLET EJECTOR SURFACE AND COATINGS” filed Mar. 25, 2022, and Provisional Patent Application No. 63/346,794 entitled “POLYMER DROPLET EJECTOR WITH SPUTTERED COATING SURFACE” filed May 27, 2022, all of which are incorporated herein by reference in their entirety.

### FIELD OF THE INVENTION

This disclosure relates to droplet generating devices, such as generating aerosols with compositions for inhalation such as by the mouth and nose.

### BACKGROUND OF THE INVENTION

The use of droplet generating devices for the delivery of substances to the respiratory system is an area of large interest. A major challenge is providing a device that delivers an accurate, consistent, and verifiable amount of substance, with a droplet size that is suitable for successful delivery of substance to the targeted area of the respiratory system.

Droplet delivery devices include an ejector mechanism with a mesh, aperture plates and like substrates having desirably sized holes and producing desirable surface contact angle that creates droplets from liquid passing through the mesh when a powered transducer acts on the liquid and ejector mechanism. In some devices a membrane may be oscillated by a powered transducer to push the liquid through the mesh and create droplets (“push mode”), while in other devices a transducer can be coupled directly to oscillate the mesh to create droplets. Examples of devices including such ejector mechanisms with substrates having apertures are described in U.S. Patent Application Pub. No. US2022/0401661 entitled “DELIVERY DEVICE WITH PUSH EJECTION” published Dec. 22, 2022, International Publication Number WO 2020/264501 entitled “DELIVERY OF SMALL DROPLETS TO THE RESPIRATORY SYSTEM VIA ELECTRONIC BREATH ACTUATED DROPLET DELIVERY DEVICE” published Dec. 30, 2020, and International Publication Number WO 2020/227717 entitled “ULTRASONIC BREATH ACTUATED RESPIRATORY DROPLET DELIVERY DEVICE AND METHODS OF USE” published Nov. 12, 2020, all of which are herein incorporated by reference in their entirety, including incorporation of such publications and patent applications as are cited and incorporated by reference or relied upon in the referenced disclosures.

Droplet delivery devices can be used for promoting inhalation of numerous therapeutic substances (e.g. pharmaceutical and medicinal) and non-therapeutic substances (e.g. nicotine and cannabinoids). Together with such substances, it can be desirable to include flavoring compositions, often oil-based, that present challenges for producing droplets capable of desirable inhalation from droplet delivery devices since the liquid compositions can have varying water and oil levels affecting interaction and droplet formation through the mesh of an ejector mechanism.

### SUMMARY OF THE INVENTION

In embodiments, the present invention includes an ejector mesh, and in some embodiment other fluid contacting parts of the ejector, that have been treated to enhance production of droplets and to achieve smaller droplet sizes in aerosols inhaled into the nose or mouth. In an embodiment, ejector parts may be treated with an oleophobic coating that reduces adhesion forces of oil-containing compositions with the ejector fluid contacting parts and the ejector mesh.

In further embodiments, the ejector mesh, and in some cases other ejector parts contacting the fluid composition, can be intentionally roughened prior to application of an oleophobic coating to further reduce the adhesion force between the additive solution and ejector mesh and parts and maintain a higher surface contact angle for forming desirable droplets from the solution/formulation.

In further embodiments, a variety of coatings and combinations of coatings can be applied after roughening of an ejector mesh, and possibly to other ejector parts contacting the fluid composition, including hydrophobic coatings (repelling water), oleophobic coatings (repelling oils or nonpolar liquids) and/or lipophobic coatings (repelling lipids or other nonpolar solvents).

In some embodiments, different ejector materials and different coatings may be utilized in varying combinations to promote better ejection of droplets from substances to be aerosolized.

In an embodiment of the invention, a polymer ejector mesh having apertures formed by laser ablation may be provided with a sputtered metal surface, such as palladium and gold metal, over the polymer mesh material. The metal sputtering adds stiffness to the polymer ejector plate to result in the unexpected effect of increased droplet ejection from a droplet ejector device.

In a further embodiment of the invention, laser ablation and/or nanomolding of various polymer materials can be used to create an ejector substrate, such as a mesh or aperture plate. In certain embodiments, polysulfone, polyimide, polyimide coated with FEP, FEP, PEEK, PTFE, PVDF, and PFA provide advantageous materials for such laser ablation or nanomolding of the ejector parts.

### DETAILED DESCRIPTION

In one embodiment of the invention, a droplet delivery device with a powered transducer includes an ejector mechanism having a mesh or similar substrate with holes of predetermined size and shape. A container of the device supplies a liquid composition to the ejector mechanism for producing droplets with a desirable size for inhalation.

In an embodiment, the liquid composition supplied to the ejector mechanism and mesh is a Nicotine formulation with 0-20% propylene glycol (PG), vegetable glycerin (VG), and glycerol (purer form of VG). These oils, typically for providing flavoring, result in a lower contact angle than through meshes having hydrophobic coatings as described in the in the incorporated disclosures identified in the Relevant Disclosures. For example, testing showed a decrease in contact angle from 111.3 to 102.8 with a 10% VG solution. It follows that the contact angle should decrease with increasing amount of additives mentioned above. In addition, contact angle should decrease by which additive is used, i.e., PG>>>glycerol>VG.

To help minimize the adhesion force between the additive solutions and the ejector material (fluid contacting parts) and ejector mesh an oleophobic and/or hydrophobic coating, or combination of coatings, is preferably used so that desirable droplets for inhalation are produced. Specifically, in one embodiment an oleophobic coating is used that allows for an aqueous solution, as well as a aqueous solution with small hydrocarbon segment additives, to have a contact angle greater than 110 degrees. In one embodiment, a hydrophobic/oleophobic monolayer may be used. Good candidates for achieving this higher contact angle in embodiments of the invention include using fluorocarbons or organofluorine compounds. Some examples of these perfluorinated compounds could include poly- and perfluoroalkyl substances (PFAS).

In some embodiments, a “universal”-type of oleophobic coating may be used to coat an ejector mesh and ejector parts contacting an oil-based composition. For example, a coating comprising a mixture of 1H,1H,2H,2H-perfluorohexyltrichlorosilane (PFTS) and n-butyl cyanoacrylate (n-BCA), combined in a dichloropentafluoropropane solution. as described at https://cen.acs.org/materials/coatings/Fluorinated-coating-utterly-repellent/96/i42 (incorporated herein by reference), may be used in embodiments of the invention. Other coatings in other embodiments may also comprise a trichlorosilane head group on a fluorinated hydrocarbon.

In some embodiments of the invention, intentionally roughing the surface of an ejector mesh, and potentially other ejector parts contacting the oil-containing formulation in other embodiments, provides better adhesion of the oleophobic coating to the desired surface. For example, it is projected that an oleophobic-coated surface without roughening will have a contact angle of approximately of 104° while roughening the surface and then coating the surface (i.e. better adhered coating) may provide a contact angle of 115°.

In various embodiments, roughening of the desired surface (such as ejector mesh and/or other ejector parts) may include roughening palladium nickel with oxygen plasma etching, argon sputter etching, ion bombardment, abrasive/roughening beads (such as aluminum oxide and zirconium oxide), other chemical etching techniques and the like. The roughened surface is then coated with an oleophobic (and preferably hydrophobic) coating to produce desired droplets from the liquid formulation of the droplet delivery device.

In other embodiments, a variety of coatings and combinations of coatings can be applied after roughening of an ejector mesh, and possibly to other ejector parts contacting the fluid composition, including hydrophobic coatings (repelling water), oleophobic coatings (repelling oils or nonpolar liquids) and/or lipophobic coatings (repelling lipids or other nonpolar solvents).

In embodiments where one or more coatings are applied to a roughened surface of an ejector mesh or part, it is preferable to apply coatings with sufficient thickness to fully cover surface peaks and valleys formed by roughening so that the coated surface is ultimately smooth as the one or more coatings adhere to the peaks and valleys.

In certain embodiments, a combination of all three of hydrophobic, oleophobic and lipophobic coatings, specifically using fluorocarbons and/or halogenated silanes may be applied to a roughened ejector mesh and/or ejector surface contacting a fluid composition of the droplet delivery devices. In order words, a coating that is lipophobic/oleophobic in addition to being hydrophobic may be applied after a desired ejector surface, e.g. ejector mesh/plates, is roughened. In some embodiments, the foregoing coatings or combined coating might not repel a liquid but provides absence/lack of attraction/adhesion between the ejector surface and liquid composition.

In further embodiments where an ejector mesh or similar substrate with apertures includes one or more coatings, the coating or coatings are also specifically applied inside the holes or apertures. Different coatings may also be applied to different surfaces an ejector mesh or parts, such as one face or surface including a first coating(s) type and an opposite face or surface including a second coating(s) type. In some embodiments, a third coating(s) type may further be applied to the holes or apertures of the ejector mesh or part.

In certain embodiments, one or more coatings may be applied to an ejector mesh or part to increase slip in order to decrease shear zone of droplets produced by the ejector mechanism. Depending on the type of fluid that is being generated into droplets, the use of different coatings or coating combinations on an ejector mesh, ejector substrate apertures and/or parts can provide better control over the shear zone and maintaining droplet sizes that are not too large or too small for the intended application of the particular kind of droplet.

In some embodiments, a polymer ejection plate increases the droplet ejection results when a metal, such as palladium or gold, is deposited, by using sputtering, evaporative deposition, or any other form of deposition, onto the surfaces of the ejection plate or part. Coatings, such as hydrophobic and oleophobic coatings and combinations thereof, can also optionally be added on to the polymer ejection plate after the metal is deposited to provide desired contact angles and further improved droplet ejection. Roughening of the deposited metal surface might also be implemented in certain embodiments and optionally utilized in combination with one or more further coatings (such as oleophobic and/or hydrophobic coatings). The metal surface deposited on a polymer mesh may provide better adhesion of surface coatings in various embodiments of the invention. The metal that deposited on the surface of the polymer mesh has a higher Young's modulus than the underlying polymer materials, therefore the sputtered/deposited metal layer on polymer material makes the aperture plate more stiff In embodiments of the invention, the aperture plate, i.e. ejector mesh/ejector plate, has apertures (in the order of microns) formed by laser ablation and the metal layer sputtered on the polymer ejector plate (on the order of nanometers) provides a metal surface layer without disrupting the apertures and liquid passage therethrough for forming droplets on the ejector plate. In some embodiments, a surface of the polymer ejection plate may be roughened and a metal layer deposited to the roughened surface.

In some embodiments, a polymer mesh having apertures formed by laser ablation may include polymer materials such as poly-methyl methacrylate, poly ether ketone, polyetherimide, polyvinylidene fluoride, ultra-high molecular weight polyethylene, polytetrafluoroethylene (PTFE), and the like. In some embodiments, a sputtered or deposited layer of metal on the polymer mesh may include a thin layer (e.g., about 30 to about 150 nm, about 60 nm to about 100 nm, about 30 nm, about 60 nm, about 80 nm, about 100 nm, etc. thick sputtering) of a precious metal, such as gold (Au), palladium (Pd), platinum (Pt), silver (Ag) and precious metal alloys. Palladium is found to be a preferably deposited/sputtered layer on polymer in embodiments of the invention. By way of example, the holes in the aperture plate (such as formed by laser ablation) may range in size from 1 micron to 6 microns, from 2 microns to 5 microns, from 3 microns to 5 microns, from 3 microns to 4 microns, and similar desirable small micron size ranges.

In a further embodiment of the invention, laser ablation and/or nanomolding of various polymer materials can be used to create an ejector substrate, such as a mesh or aperture plate. In certain embodiments, polysulfone, polyimide, polyimide coated with fluorinated ethylene propylene (FEP), fluorinated ethylene propylene (FEP), polyether ether ketone (PEEK), polytetrafluoroethylene (PTFE), polyvinylidene fluoride or polyvinylidene difluoride (PVDF), and perfluoroalkoxy alkane (PFA) provide advantageous materials for such laser ablation or nanomolding of the ejector parts.

In one aspect of the invention, a droplet delivery device includes an ejector comprising a polymer mesh with apertures and a deposited metal layer on the polymer mesh. In one embodiment a metal layer is sputtered on the polymer mesh, but other deposition methods are encompassed for depositing the metal layer on a mesh of the invention.

In a further aspect, the apertures of a droplet delivery device are formed by laser ablation.

In a further aspect, a metal layer deposited on an ejector mesh of the invention includes one or more of gold, palladium, platinum, and silver.

In a further aspect, a metal layer deposited on an ejector mesh of the invention includes one or more precious metal alloys.

In a further aspect, an ejector mesh of the invention includes one or more of poly-methyl methacrylate, polyether ether ketone, polyetherimide, polyvinylidene fluoride, ultra-high molecular weight polyethylene, polysulfone, polyimide, fluorinated ethylene propylene, perfluoroalkoxy alkane and polytetrafluoroethylene.

In aspects of the invention, a polymer mesh is nano molded.

In another aspect of the invention, an oleophobic or hydrophobic coating is applied on the metal layer. In another aspect of the invention, a metal layer may be roughened to better adhere a coating, such as an oleophobic or hydrophobic coating.

In certain aspects of the invention, a metal layer of an ejector mesh is roughened to include peaks and valleys and one or more coatings cover the peaks and valleys of the metal layer to provide a smooth surface.

In various aspects of the invention, at least one coating is applied in a plurality of apertures of an ejector mesh, such as a polymer mesh.

In another aspect of the invention, a droplet delivery device including an ejector comprises a substrate with apertures, a transducer, such as a powered piezoelectric transducer, configured to vibrate the substrate, a liquid supply to the substrate, and one or more coatings applied to a roughened surface of the substrate with apertures. In one aspect, the substrate is a polymer. In one aspect, one or more coatings of the substrate include an oleophobic coating. In another aspect, one or more coatings of the substrate include a deposited metal. In another aspect, one or more coatings of the substrate include a hydrophobic coating.

While the invention has been described with reference to exemplary embodiments, it will be understood by those skilled m the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed:

1. A droplet delivery device including an ejector comprising:
a polymer mesh with apertures; and
a deposited metal layer on the polymer mesh.

2. The droplet delivery device of claim 1, wherein the apertures are formed by laser ablation.

3. The droplet delivery device of claim 1, wherein the metal layer is sputtered on the polymer mesh.

4. The droplet delivery device of claim 3, wherein the metal layer includes one or more of gold, palladium, platinum, and silver.

5. The droplet delivery device of claim 4, wherein the metal layer includes one or more precious metal alloys.

6. The droplet delivery device of claim 2, wherein the metal layer includes one or more of gold, palladium, platinum, and silver.

7. The droplet delivery device of claim 1, wherein the metal layer includes one or more of gold, palladium, platinum, and silver.

8. The droplet delivery device of claim 7, wherein the polymer mesh includes one or more of poly-methyl methacrylate, polyether ether ketone, polyetherimide, polyvinylidene fluoride, ultra-high molecular weight polyethylene, polysulfone, polyimide, fluorinated ethylene propylene, perfluoroalkoxy alkane and polytetrafluoroethylene.

9. The droplet delivery device of claim 1, wherein the polymer mesh is nano molded.

10. The droplet delivery device of claim 1, further comprising an oleophobic or hydrophobic coating on the metal layer.

11. The droplet delivery device of claim 1, further comprising a hydrophobic coating on the metal layer.

12. The droplet delivery device of claim 11, wherein the metal layer is roughened.

13. The droplet delivery device of claim 10, wherein the metal layer is roughened.

14. The droplet delivery device of claim 1, wherein the metal layer is roughened to include peaks and valleys and one or more coatings cover the peaks and valleys of the metal layer to provide a smooth surface.

15. The droplet delivery device of claim 15, further comprising at least one coating applied in a plurality of apertures of the polymer mesh.

16. A droplet delivery device including an ejector comprising:
a substrate with apertures;
a transducer configured to vibrate the substrate;
a liquid supply to the substrate; and
one or more coatings applied to a roughened surface of the substrate.

17. The droplet delivery device of claim 16, wherein the substrate is a polymer.

18. The droplet delivery device of claim 16, wherein the one or more coatings include an oleophobic coating.

19. The droplet delivery device of claim 16, wherein the one or more coatings include a deposited metal.

20. The droplet delivery device of claim 16, wherein the one or more coatings include a hydrophobic coating.
